(19) United States
(12) Patent Application Publication
(10) Pub. No.: US 2026/0091171 A1
(43) Pub. Date: Apr. 2, 2026

(54) Syringe Assembly For Administering Multiple Fluids

(71) Applicant: Abraham C. Ng, American Canyon, CA (US)

(72) Inventor: Abraham C. Ng, American Canyon, CA (US)

(21) Appl. No.: 18/902,936

(22) Filed: Oct. 1, 2024

(51) Int. Cl. A61M 5/168; A61M 5/315

(52) U.S. Cl. CPC A61M 5/16827; A61M 5/31511

(57) ABSTRACT

A framework for administering multiple fluids using a single syringe assembly. The syringe assembly includes a barrel and first and second plungers. The barrel includes a first inside surface defining a first fluid chamber for retaining a first fluid and a distal tip defining a first passageway in fluid communication with the first fluid chamber. The first plunger includes a first stopper and a second inside surface defining a second fluid chamber for retaining a second fluid, wherein the first stopper is slidably positioned in fluid-tight engagement with the first inside surface of the barrel for driving the first fluid from the first fluid chamber though the first passageway. The second plunger includes a second stopper that is slidably positioned in fluid tight engagement with the second inside surface of the first plunger for driving the second fluid from the second fluid chamber.

### TECHNICAL FIELD

The present disclosure generally relates to syringe assemblies, and more particularly to a syringe assembly for administering multiple fluids.

### BACKGROUND

An intravenous (IV) catheter (e.g., peripheral IV) is a therapeutic device commonly used to administer treatments, such as medications, blood transfusions and liquid nutrition. After a provider inserts an IV catheter into a vein of the patient, it can remain in place for several days, which prevents the need for repeated needle sticks. A given tubing line set of the IV catheter may be repeatedly employed during the course of extended medication therapy. A number of tubing line sets may be successively employed. For example, it is typical to replace a given tubing line set every two or three days. During extended therapy applications, it is common practice to disconnect the IV catheter from a fluid source and tubing line set between infusions.

In conjunction with the repeated connection/disconnection of the IV catheter, it is usual practice to purge the IV catheter with a flush solution (e.g. a saline solution) after infusion of a given liquid medication. Post infusion flushing not only flushes through any remaining liquid medication to achieve the desired therapeutic effect, but also reduces any chance that the IV catheter may become blocked in-between infusions, e.g. by a blood clot that may otherwise form in the IV catheter.

After medication is infused, a unit dose syringe may be used to administer the flush solution. To use a unit dose syringe, medical personnel generally remove the syringe from packaging, removes a cap from the syringe, removes any air in the syringe, swabs a vascular catheter access port with an antibacterial material, interconnects the syringe to a vascular catheter access port, advances the syringe plunger to infuse the flush solution, removes the syringe from the vascular catheter access port and discards the used syringe with its wrapper. As may be appreciated, such steps may need to be repeated numerous times over the course of extended medication therapy, thereby entailing significant medical personnel time and resulting in substantial medical waste and time.

### SUMMARY

Described herein is a framework for administering multiple fluids using a single syringe assembly. The syringe assembly includes a barrel and first and second plungers. The barrel includes a first inside surface defining a first fluid chamber for retaining a first fluid and a distal tip defining a first passageway in fluid communication with the first fluid chamber. The first plunger includes a first stopper and a second inside surface defining a second fluid chamber for retaining a second fluid, wherein the first stopper is slidably positioned in fluid-tight engagement with the first inside surface of the barrel for driving the first fluid from the first fluid chamber through the first passageway. The second plunger includes a second stopper that is slidably positioned in fluid tight engagement with the second inside surface of the first plunger for driving the second fluid from the second fluid chamber.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings.

FIG. 1a is a cross-sectional side view of an exemplary syringe assembly prior to an infusion procedure;

FIG. 2 is a cross-sectional top view and side view of an exemplary barrel;

FIG. 3 is a cross-sectional side view of an exemplary first plunger;

FIG. 4 is a cross-sectional side view of an exemplary second plunger;

FIG. 5a is a top view of an exemplary syringe assembly;

FIG. 5b is a bottom view of an exemplary syringe assembly; and

FIG. 6 is an exemplary method of administering fluids using a single syringe assembly.

### DETAILED DESCRIPTION

In the following description, numerous specific details are set forth such as examples of specific components, devices, methods, etc., in order to provide a thorough understanding of implementations of the present framework. It will be apparent, however, to one skilled in the art that these specific details need not be employed to practice implementations of the present framework. In other instances, well-known materials or methods have not been described in detail in order to avoid unnecessarily obscuring implementations of the present framework. While the present framework is susceptible to various modifications and alternative forms, specific embodiments thereof are shown by way of example in the drawings and will herein be described in detail. It should be understood, however, that there is no intent to limit the invention to the particular forms disclosed, but on the contrary, the intention is to cover all modifications, equivalents, and alternatives falling within the spirit and scope of the invention. Furthermore, for ease of understanding, certain method steps are delineated as separate steps; however, these separately delineated steps should not be construed as necessarily order dependent in their performance.

A fluid delivery framework is presented herein. The fluid delivery framework includes a syringe assembly with a first plunger for intravenously administering a first fluid contained in a first fluid chamber and second plunger for intravenously administering a second fluid contained in a second fluid chamber. The first fluid may be a liquid medication or drug, while the second fluid may be a flush solution. By administering two different types of fluids using a single syringe without disconnecting from the vascular catheter access port, the delay of care and risk of infection are reduced significantly. The present syringe assembly provides a more effective, economical and convenient method of intravenous (IV) administration. It is easier to use and more effective during, for example, emergency cases, rescue operations and other time-sensitive scenarios. These and other exemplary advantages and features will be described in more details in the following description.

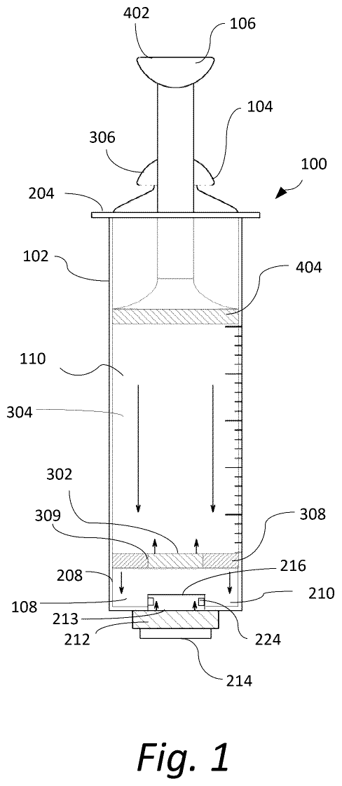

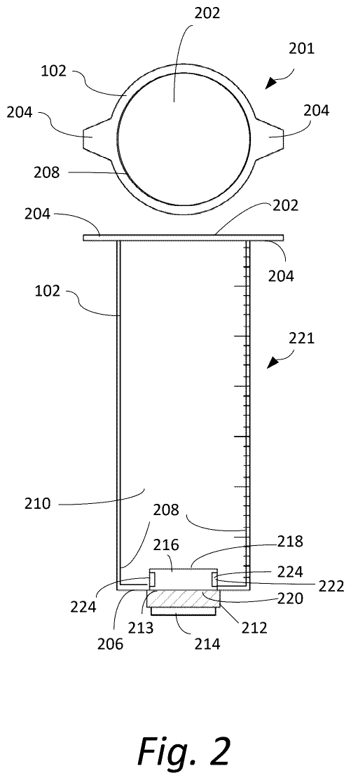

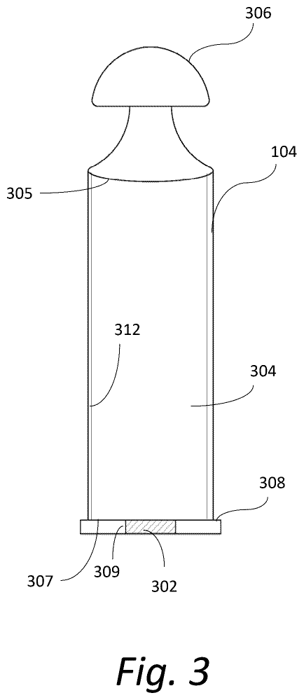

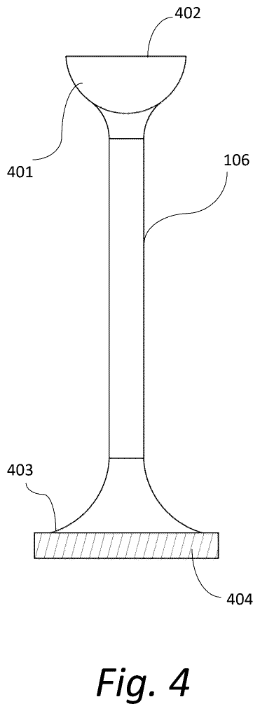

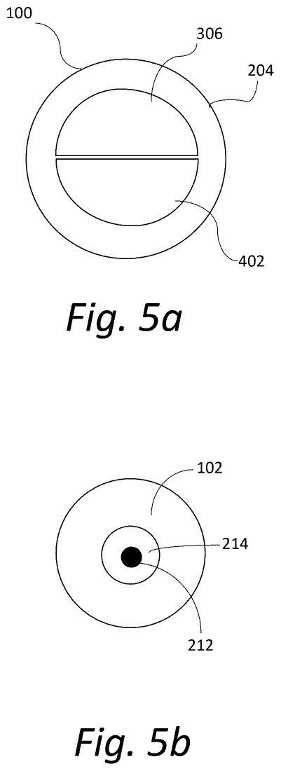

FIG. 1 is a cross-sectional side view of an exemplary syringe assembly 100 prior to an infusion procedure. The syringe assembly 100 includes a barrel 102, first plunger 104 and second plunger 106. First plunger 104 includes a first stopper 308 that is slidably positioned in fluid-tight engagement with inside surface 208 of barrel 102 for driving first fluid 108 out of first fluid chamber 210 in the barrel 102. Second plunger 106 includes a second stopper 404 that is slidably positioned in fluid-tight engagement with inside surface 312 of first plunger 104 for driving second fluid 110 out of second fluid chamber 304. FIG. 2 is a cross-sectional top view 201 and side view 221 of an exemplary barrel 102. FIG. 3 is a cross-sectional side view of an exemplary first plunger 104. FIG. 4 is a cross-sectional side view of an exemplary second plunger 106. FIG. 5a is a top view of an exemplary syringe assembly 100; and FIG. 5b is a bottom view of an exemplary syringe assembly 100.

Barrel 102 has a generally elongate body including an open proximal end 202 for receiving first plunger 104. Barrel 102 may further include a barrel flange 204, a distal end 206 and an inside surface 208 defining a first fluid chamber 210 for retaining first fluid 108. Distal end 206 includes a tip 212 extending distally and having a passageway 213 therethrough in fluid communication with first fluid chamber 210. Distal end 206 may include a cylindrical luer lock 214 concentrically surrounding tip 212. Luer lock 214 is a screw connection that creates a leak-free seal and maintains a continuous lumen for fluid communication between the passageway 213 and a downstream IV line or needle for administering fluids to a patient.

Distal end 206 further includes a protrusion 216 disposed adjacent to the passageway 213 in tip 212. Protrusion 216 may cover a proximal opening of passageway 213. Protrusion 216 is generally cylindrical or disc-shaped to conform with the inside surface of passageway 309. Protrusion 216 includes a proximal surface 218, a distal surface 220 and a curved side surface 222. Proximal surface 218 may be substantially flat. Alternatively, proximal surface 218 is cone-shaped or tapered with the central portion extending upwards. Other types of surfaces are also possible. Side surface 222 includes channels 224 and proximal surface 218 include additional channels that pass to distal surface 220 to allow communication of fluid from first fluid chamber 210 to the passageway 213 in the tip 212. Protrusion 216 is made of a hard material (e.g., plastic). Protrusion 216 of barrel 102 serves to detach or displace plug 302 of the first plunger 104 to allow communication of second fluid 110 from second fluid chamber 304 to passageway 213 in tip 212.

First plunger 104 has a generally elongate body including an open proximal end 305 for receiving second plunger 106. First plunger 104 may further include a first plunger flange 306, a distal end 307 and an inside surface 312 defining a second fluid chamber 304 for retaining second fluid 110. A flexible stopper 308 is disposed on the distal end of first plunger 106. Stopper 308 includes any flexible resilient material (e.g., thermoplastic elastomers, natural rubber, synthetic rubber) suitable for providing sealing characteristics while under compression. Stopper 308 is slidably positioned in fluid-tight engagement with the inside surface 208 of the barrel 102 for drawing first fluid 108 out of first fluid chamber 210 by movement of the stopper 308 relative to the barrel 102.

In some implementations, stopper 308 defines a passageway 309 therethrough that is temporarily sealed by a detachable plug 302. Detachable plug 302 is slidably positioned in fluid-tight engagement with an inside surface of passageway 309. Detachable plug 302 may be generally cylindrical or disc-shaped to conform to the inside surface of passageway 309. Detachable plug 302 is made of any flexible resilient material (e.g., thermoplastic elastomers, natural rubber, synthetic rubber). Detachable plug 302 may be displaced in response pressure being applied to first plunger flange 306, which pushes the detachable plug 302 against protrusion 216 disposed on the distal end of 206 of barrel 102.

In some implementations, protrusion 216 is generally cylindrical or disc-shaped to conform with the inside surface of passageway 309. The width of protrusion 216 is smaller or substantially equal to the width of detachable plug 302 to fit into passageway 309. The height of protrusion 216 is taller or substantially equal to the height of passageway 309 to ensure that the detachable plug 302 is completely displaced from passageway 309 and freely floating in second fluid 110 without obstructing the passageway 309. After the plug 302 is displaced, second fluid 110 flows from second fluid chamber 304 through second passageway 309 and protrusion 216 and into passageway 213 in tip 212.

Second plunger 106 has a generally elongate body including a proximal end 401 having a second plunger flange 402 and a distal end 403. In some implementations, as illustrated by the top view of the syringe assembly 100 shown in FIG. 5a, the first plunger flange 306 and second plunger flange 402 together form a circular shape within the circular shape of the barrel flange 204.

A flexible stopper 404 is disposed on the distal end 403 of second plunger 106. Stopper 404 includes any flexible resilient material suitable for providing sealing characteristics while under compression. For example, stopper 404 may be made of thermoplastic elastomers, natural rubber, synthetic rubber or the like. Stopper 404 is slidably positioned in fluid-tight engagement with the inside surface 312 of first plunger 104 for driving second fluid 110 out of second fluid chamber 304 through passageway 309, protrusion 216 and into passageway 213 in tip 212 by movement of the stopper 404 relative to the first plunger 104.

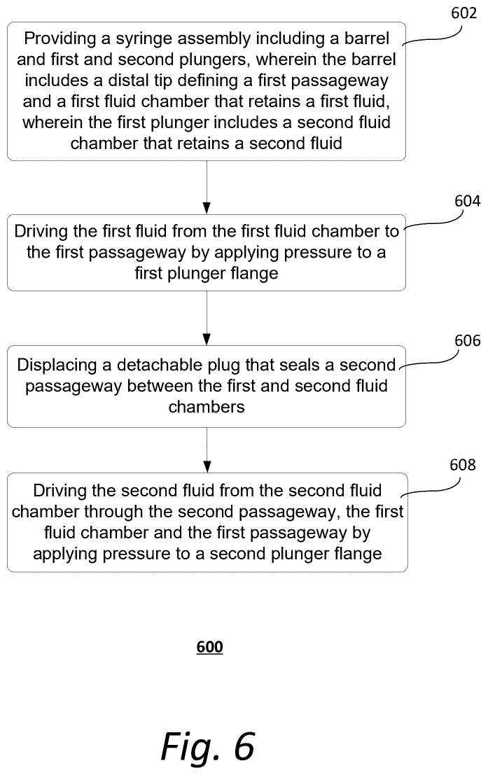

FIG. 6 shows an exemplary method 600 of administering fluids using a single syringe assembly. It should be understood that the steps of the method 600 may be performed in the order shown or a different order. Additional, different, or fewer steps may also be provided. Further, the method 600 may be implemented with syringe assembly 100 of FIGS. 1-5b, a different system, or a combination thereof.

At 602, a syringe assembly 100 including a barrel 102, first plunger 104 and second plunger 106 is provided. Barrel 102 includes a distal tip 212 defining a passageway 213 therethrough and an inside surface 208 that defines a first fluid chamber 210 for retaining a first fluid 108. First plunger 104 includes an inside surface 312 that defines a second fluid chamber 304 for retaining a second fluid 110. A user may connect the distal tip 212 of the barrel 102 to an IV line or needle via a luer lock 214 disposed on barrel 102.

In some implementations, the syringe assembly 110 is pre-filled during manufacturing of the syringe assembly 100 via a sterile filling method. The first fluid chamber 210 may be pre-filled with first fluid 108 and the second fluid chamber 304 may be pre-filled with second fluid 110. In some implementations, first fluid 108 is a liquid medication or drug (e.g., diluted or undiluted) and second fluid 110 is a flush solution intended for flushing. The flush solution may be, for example, a saline flush solution, dextrose or sterile water.

At 604, first fluid 108 is driven from the first fluid chamber 210 to the first passageway 213 by applying pressure to first plunger flange 306 of first plunger 104. More particularly, barrel 102 may be held (or gripped) via barrel flange 204. Pressure may then be applied to first plunger flange 306 by, for example, a user's thumb, in the distal direction. This moves the stopper 308 on first plunger 104 relative to barrel 102, forcing the first fluid 108 in first fluid chamber 210 out of the first fluid chamber 210 and into passageway 213 in tip 212 and through any downstream IV line connected via the luer lock 214.

At 606, a detachable plug 302 is displaced. Detachable plug 302 temporarily seals a second passageway 309 between the first fluid chamber 210 and the second fluid chamber 304. The second passageway 309 extends through a distal stopper 308 of the first plunger 104. Detachable plug 302 may be displaced by applying additional pressure to first plunger 306, which pushes the plug 302 against a protrusion 216 disposed on the distal end 206 of barrel 102. Detachable plug 302 is completely displaced from passageway 309 by protrusion 216 and freely floats in second fluid 110 without obstructing the passageway 309. After the plug 302 is displaced, second fluid chamber 1010 is in fluid communication with passageway 213 in tip 212.

At 608, second fluid 110 from the second fluid chamber 304 is driven through second passageway 309, first fluid chamber 210 and first passageway 213 by applying pressure to a second plunger flange 402 of second plunger 106. Pressure may be applied the second plunger flange 402 by, for example, the user's thumb moving in the distal direction. This moves the stopper 404 of second plunger 106 relative to the first plunger 104, forcing the second fluid 110 in second fluid chamber 304 out of the second fluid chamber 304, through protrusion 216 and into passageway 213 in tip 212 and through any downstream IV line connected to the tip 212 via the luer lock 214.

While the present framework has been described in detail with reference to exemplary embodiments, those skilled in the art will appreciate that various modifications and substitutions can be made thereto without departing from the spirit and scope of the invention as set forth in the appended claims. For example, elements and/or features of different exemplary embodiments may be combined with each other and/or substituted for each other within the scope of this disclosure and appended claims.

What is claimed is:

1. A syringe assembly, comprising:
a barrel including a first inside surface defining a first fluid chamber for retaining a first fluid and a distal tip defining a first passageway in fluid communication with the first fluid chamber;
a first plunger including a first stopper and a second inside surface defining a second fluid chamber for retaining a second fluid, wherein the first stopper is slidably positioned in fluid-tight engagement with the first inside surface of the barrel for driving the first fluid from the first fluid chamber though the first passageway; and
a second plunger including a second stopper that is slidably positioned in fluid tight engagement with the second inside surface of the first plunger for driving the second fluid from the second fluid chamber.

2. The syringe assembly of claim 1 wherein the barrel further comprises a protrusion disposed adjacent to the first passageway to displace a detachable plug positioned in the first stopper.

3. The syringe assembly of claim 2 wherein the protrusion includes a substantially flat proximal surface.

4. The syringe assembly of claim 2 wherein the protrusion includes channels that pass from proximal and side surfaces of the protrusion to a distal surface of the protrusion, wherein the channels are in fluid communication with the first fluid chamber and the first passageway.

5. The syringe assembly of claim 2 wherein the protrusion comprises a cylindrical or disc shape.

6. The syringe assembly of claim 2 wherein the detachable plug is slidably positioned in fluid-tight engagement with a third inside surface of a second passageway defined through the first stopper.

7. The syringe assembly of claim 6 wherein the second plunger drives the second fluid from the second fluid chamber through the second passageway and the protrusion into the first passageway in response to the second stopper moving relative to the first plunger.

8. The syringe assembly of claim 6 wherein the detachable plug floats in the second fluid after being displaced by the protrusion.

9. The syringe assembly of claim 7 wherein the detachable plug comprises a cylindrical or disc shape.

10. The syringe assembly of claim 1 wherein the first fluid comprises a medication liquid.

11. The syringe assembly of claim 1 wherein the second fluid comprises a flush solution.

12. The syringe assembly of claim 11 wherein the flush solution comprises a saline flush solution, dextrose or sterile water.

13. A method of administering fluids, comprising:
providing a syringe assembly including a barrel and first and second plungers, wherein the barrel includes a distal tip that defines a first passageway therethrough and a first fluid chamber that retains a first fluid, wherein the first plunger includes a first stopper and a second fluid chamber that retains a second fluid;
driving the first fluid from the first fluid chamber through the first passageway by applying pressure to a first plunger flange of the first plunger;
displacing a detachable plug that seals a second passageway defined in the first stopper; and
driving the second fluid from the second fluid chamber though the second passageway and the first passageway by applying pressure to a second plunger flange of the second plunger.

14. The method of claim 13 further comprising pre-filling the first and second fluid chambers with the first and second fluids during manufacturing of the syringe assembly.

15. The method of claim 13 wherein applying pressure to the first plunger flange of the first plunger moves a first stopper slidably positioned in fluid-tight engagement with a first inside surface of the barrel.

16. The method of claim 13 wherein displacing the detachable plug comprises applying pressure to the first plunger, wherein the first plunger pushes the detachable plug against a protrusion disposed on a distal end of the barrel.

17. The method of claim 13 wherein applying pressure to the second plunger flange of the second plunger moves a second stopper that is slidably positioned in fluid tight engagement with a second inside surface of the first plunger.

18. The method of claim 13 wherein driving the first fluid from the first fluid chamber comprises driving a medication liquid from the first fluid chamber.

19. The method of claim 13 wherein driving the second fluid from the second fluid chamber comprises driving a flush solution from the second fluid chamber.

20. The method of claim 19 wherein driving the flush solution from the second fluid chamber comprises driving a saline flush solution, dextrose or sterile water from the second fluid chamber.
